(19) United States
(12) Patent Application Publication
(10) Pub. No.: US 2014/0112913 A1
(43) Pub. Date: Apr. 24, 2014

(54) METHODS OF PROGNOSING AND ADMINISTERING TREATMENT FOR INFLAMMATORY DISORDERS

(71) Applicant: Jonathan Mirich, Des Moines, WA (US)

(72) Inventor: Jonathan Mirich, Des Moines, WA (US)

(73) Assignee: Genqual Corporation, Des Moines, WA (US)

(21) Appl. No.: 13/985,025

(22) PCT Filed: Feb. 9, 2012
(86) PCT No.: PCT/US12/24448

§ 371 Date: Jan. 3, 2014

Related U.S. Application Data
(60) Provisional application No. 61/441,516, filed on Feb. 10, 2011.

(51) Int. Cl. C12Q 1/68

(52) U.S. Cl. CPC C12Q 1/6883
USPC 424/133.1; 435/6.11; 424/158.1

(57) ABSTRACT

The present invention provides methods for determining responsiveness to an IL-6 inhibitor JAK/STAT pathway inhibitor in a subject. The present invention also provides methods for treating disease with an IL-6 inhibitor or a JAK/STAT pathway inhibitor.

### CROSS-REFERENCE

This application claims the priority of U.S. Patent Application Ser. No. 61/441,516, filed Feb. 10, 2011, which is incorporated herein by reference in its entirety.

### BACKGROUND OF THE INVENTION

Approximately 90% of all polymorphisms in the human genome are single nucleotide polymorphisms (SNPs). SNPs are single base pair positions in DNA at which different alleles, or alternative nucleotides, exist in some population. An individual may be homozygous or heterozygous for an allele at each SNP position. A SNP may arise due to a substitution of one nucleotide for another at the polymorphic site. Substitutions can be transitions or transversions. A transition is the replacement of one purine nucleotide by another purine nucleotide, or one pyrimidine by another pyrimidine. A transversion is the replacement of a purine by a pyrimidine, or vice versa. A SNP may also be a single base insertion/deletion variant. A substitution that changes a codon coding for one amino acid to a codon coding for a different amino acid is referred to as a non-synonymous codon change, or missense mutation. A synonymous codon change, or silent mutation, is one that does not result in a change amino acid due to the degeneracy of the genetic code. A nonsense mutation is a type of non-synonymous codon change that results in the formation of a stop codon, thereby leading to premature termination of a polypeptide chain and a defective protein.

SNPs may produce alterations in gene expression or in the expression or function of a gene product. Such SNPs do not necessarily have to occur in coding regions but rather are sometimes found in promoter regions, intron-exon boundaries that may perturb splicing, or in mRNA processing signal sequences. As a result of these alterations, some SNPs have been shown to be predictive of a possible clinical phenotype. For example, mutational status of KRAS has been shown to be useful marker for predicting survival in patients with metastatic colorectal cancer treated with cetuximab (Ann Oncol. 2008 March; 19(3):508-15). Genetic variability in the VKORC1 and CYP2C9 genes has also been found to be associated with increased warfarin sensitivity, and examining the sequences of these genes has been described for use in predicting warfarin dosage (U.S. application Ser. No. 11/757,860).

Current treatment options for RA and other inflammatory disorders are incomplete and have significant drawbacks. Disease modifying anti-rheumatic drugs (DMARDS) such as methotrexate, while effective are chemotherapeutic in nature. Side effects such as nausea and permanent liver damage make long term use undesirable. Actemra (tocilizumab, an anti-IL-6 humanized mAb against soluble and cellular IL-6 receptor) was initially approved in early January 2010 for the treatment of rheumatoid arthritis for patients who failed adequate treatment with other approved therapies. Actemra has a failure rate due to side effects or lack of efficacy of around one-third of patients. Similar lack of effectiveness and undesirable side effects have been observed with treatments directed to inhibition of tumor necrosis factor alpha (TNF-alpha). Clearly, there is a need in the art for methods of determining responsiveness to treatment, to improve efficacy rate and to avoid incurring damage from side effects in cases where patients are unlikely to respond to the treatment.

### SUMMARY OF THE INVENTION

In one aspect, the present invention provides a method for determining responsiveness to an IL-6 inhibitor in a subject, comprising: (a) assaying SNP rs1800795 for presence of G/C or G/G allele in a subject in need of a treatment of a disease with an IL-6 inhibitor; and (b) determining the responsiveness of said subject to said IL-6 inhibitor treatment, based on the results from step (a).

In another aspect, the present invention provides a method for treating a subject, comprising: (a) assaying SNP rs1800795 in a subject in need of a treatment of a disease with an IL-6 inhibitor; and (b) administering to said subject a pharmaceutically effective amount of said IL-6 inhibitor, based on the allele status of SNP rs180079.

In some embodiments, the disease is selected from the group consisting of: rheumatoid arthritis, chronic juvenile arthritis, Crohn's disease, autoimmune diseases, diabetes and insulin sensitivity, neuroblastoma, systemic lupus erythematosus (SLE or lupus), and solid tumor cancer. In some embodiments, the IL-6 inhibitor comprises an antibody against IL-6 or a fragment thereof, such as tocilizumab.

In another aspect, the present invention provides a method for determining responsiveness to a JAK/STAT pathway inhibitor in a subject, comprising: (a) assaying SNP rs7574865 for presence of G/T or T/T/ allele in a subject in need of a treatment of a disease with a JAK/STAT pathway inhibitor; and (b) determining the responsiveness of said subject to said JAK/STAT pathway inhibitor treatment based on the results from step (a).

In yet another aspect, the present invention provides a method for treating a subject, comprising: (a) assaying SNP rs7574865 in a subject in need of a treatment of a disease with a JAK/STAT pathway inhibitor; and (b) administering to said subject a pharmaceutically effective amount of said JAK/STAT pathway inhibitor in a dose dependent on results from step (a).

In some embodiments, the disease is selected from the group consisting of: rheumatoid arthritis, chronic juvenile arthritis, Crohn's disease, systemic lupus erythmatosus (SLE), Sjorgen's syndrome, and associated inflammatory disease. In some embodiments, the JAK/STAT pathway inhibitor comprises an antibody against TNF, TNF-receptor (soluble and/or cellular), IL-6, IL-6 receptor (soluble and/or cellular), INF-alpha, JAK2 or JAK3, or a fragment thereof, such as Humira or Remicade. In some embodiments, the inhibitors comprises a protein or peptide that binds to TNF, TNF-receptor (soluble and/or cellular), IL-6, IL-6 receptor (soluble and/or cellular), INF-alpha, JAK2 or JAK3, preferably acting as a dominant negative inhibitor (e.g. Enbrel).

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

In one aspect, the present disclosure relates generally to the use of SNPs in determining responsiveness to a treatment of an inflammatory disorder. In some embodiments, the SNP is rs1800795 and the treatment is inhibition of IL-6. In some embodiments, the SNP is rs7574865 and the treatment is inhibition of the JAK/STAT signaling pathway. The present disclosure also involves methods of treatment of diseases wherein a SNP is assayed and the dosing regimen of the treatment is based on the allele status of the SNP.

### SNP Detection

SNPs show great promise as markers for a number of phenotypic traits, non-limiting examples of which include disease propensity and severity, wellness propensity, drug responsiveness, and susceptibility to adverse drug reactions. Knowledge of the association of a particular SNP with a phenotypic trait, coupled with the knowledge of whether an individual has said particular SNP, can enable the targeting of diagnostic, preventative and therapeutic applications to allow better disease management, to enhance understanding of disease states and to ultimately facilitate the discovery of more effective treatments, such as personalized treatment regimens.

Indeed, a number of databases have been constructed of known SNPs, and for some such SNPs, the biological effect associated with a SNP. For example, the NCBI SNP database “dbSNP” is incorporated into NCBI's Entrez system and can be queried using the same approach as the other Entrez databases such as PubMed and GenBank. This database has records for over 1.5 million SNPs mapped onto the human genome sequence. Each dbSNP entry includes the sequence context of the polymorphism (i.e., the surrounding sequence), the occurrence frequency of the polymorphism (by population or individual), and the experimental methods), protocols, and conditions used to assay the variation, and can include information associating a SNP with a particular phenotypic trait.

At least in part because of the potential impact on health and wellness, there has been and continues to be a great deal of effort to develop methods that reliably and rapidly identify SNPs. This is no trivial task, at least in part because of the complexity of human genomic DNA, with a haploid genome of 3×10̂9 base pairs, and the associated sensitivity and discriminatory requirements.

Genotyping approaches to detect SNPs well-known in the art include DNA sequencing, methods that require allele specific hybridization of primers or probes, allele specific incorporation of nucleotides to primers bound close to or adjacent to the polymorphisms (often referred to as “single base extension”, or “mini sequencing”), allele-specific ligation (joining) of oligonucleotides (ligation chain reaction or ligation padlock probes), allele-specific cleavage of oligonucleotides or PCR products by restriction enzymes (restriction fragment length polymorphisms analysis or RFLP) or chemical or other agents, resolution of allele-dependent differences in electrophoretic or chromatographic mobilities, by structure specific enzymes including invasive structure specific enzymes, or mass spectrometry. Analysis of amino acid variation is also possible where the SNP lies in a coding region and results in an amino acid change.

DNA sequencing allows the direct determination and identification of SNPs. The benefits in specificity and accuracy are generally outweighed for screening purposes by the difficulties inherent in whole genome, or even targeted sub-genome, sequencing. Mini-sequencing involves allowing a primer to hybridize to the DNA sequence adjacent to the SNP site on the test sample under investigation. The primer is extended by one nucleotide using all four differentially tagged fluorescent dideoxynucleotides (A, C, G, or T), and a DNA polymerase. Only one of the four nucleotides (homozygous case) or two of the four nucleotides (heterozygous case) is incorporated. The base that is incorporated is complementary to the nucleotide at the SNP position.

A number of methods currently used for SNP detection involve site-specific and/or allele-specific hybridization (Matsuzaki, H. et al. Genome Res. 14:414-425 (2004); Matsuzaki, H. et al. Nat. Methods 1:109-111 (2004); Sethi, A. A. et al. Clin. Chern. 50(2):443-446 (2004), each of the foregoing is herein incorporated by reference in its entirety). These methods are largely reliant on the discriminatory binding of oligonucleotides to target sequences containing the SNP of interest. The techniques of Affymetrix (Santa Clara, Calif.) and Nanogen Inc. (San Diego, Calif.) are particularly well-known, and utilize the fact that DNA duplexes containing single base mismatches are much less stable than duplexes that are perfectly base-paired. The presence of a matched duplex is detected by fluorescence.

Examples of DNA microarrays include those commercially available from Affymetrix, Inc. (Santa Clara, Calif.), including the GeneChip™ Mapping Arrays including Mapping 100K Set, Mapping 10K 2.0 Array, Mapping 10K Array, Mapping 500K Array Set, and GeneChip™ Human Mitochondria' Resequencing Array 2.0. The Mapping 10K array, Mapping 100K array set, and Mapping 500K array set interrogate more than 10,000, 100,000 and 500,000 different human SNPs, respectively. SNP detection and analysis using GeneChip™ Mapping Arrays is described in part in Kennedy, G. C., et al., Nature Biotechnology 21, 1233-1237, 2003; Liu, W. M., Bioinformatics 19, 2397-2403, 2003; as well as in U.S. Pat. Nos. 5,445,934; 5,744,305; 6,261,776; 6,291,183; 5,799,637; 5,945,334; 6,346,413; 6,399,365; and 6,610,482, and EP 619 321; 373 203.

In some embodiments, genomic DNA is obtained from a subject. The genomic DNA can be digested with a restriction enzyme, such as XbaI or Hind III. Other DNA microarrays may be designed for use with other restriction enzymes, e.g., Sty I or NspI. Fragments resulting from the digestion can be ligated on both ends with an adapter sequence that recognizes the overhangs from the restriction digest. Subsequently, fragments having the adapter sequence at both ends can be amplified using a generic primer that recognizes the adapter sequence. The PCR conditions used for amplification may be optimized to amplify fragments that have a unique length, e.g., between 250 and 2,000 base pairs in length. In some embodiments, amplified DNA sequences are fragmented, labeled and hybridized with the DNA microarray (e.g., 100K Set Array or other array). Hybridization can be followed by a step of washing and staining Results can be visualized using a scanner that enables the viewing of intensity of data collected and a software “calls” the bases present at each of the SNP positions interrogated. Computer implemented methods for determining genotype using data from mapping arrays are disclosed, for example, in Liu, et al., Bioinformatics 19:2397-2403, 2003; and Di et al., Bioinformatics 21:1958-63, 2005.

In some embodiments, SNP detection is carried out using PCR methods. Tetra-primer ARMS-PCR employs two pairs of primers to amplify two alleles in one PCR reaction. The primers are designed such that the two primer pairs overlap at a SNP location but each match perfectly to only one of the possible SNPs. As a result, if a given allele is present in the PCR reaction, the primer pair specific to that allele will produce product but not to the alternative allele with a different SNP. The two primer pairs are also designed such that their PCR products are of a significantly different length allowing for easily distinguishable bands by gel electrophoresis. In examining the results, if a genomic sample is homozygous, then the PCR products that result will be from the primer which matches the SNP location to the outer, opposite strand primer as well from the two opposite, outer primers. If the genomic sample is heterozygous, then products will result from the primer of each allele to their respective outer primer counterparts as well as from the two opposite, outer primers.

PCR can also be carried out using bi-functional primer molecules in which a primer is covalently linked to a probe. The probe can comprise a fluorophore-quencher pair. In the absence of the target (e.g., a particular SNP allele), the quencher nearly absorbs the fluorescence emitted by the fluorophore. During a productive PCR reaction, in the presence of the target, the fluorophore and the quencher separate which leads to an increase in the fluorescence emitted. The fluorescence can be detected and measured in the reaction tube. Multiple SNP alleles can be detected in this manner by utilizing bi-functional primers that target different alleles and comprise fluorophores of different colors.

In some embodiments, linear-after-the-exponential-PCR (LATE-PCR) is employed to detect SNPs. LATE-PCR (similar to Asymmetric PCR) utilizes a limiting primer with a higher melting temperature (Tm) than the excess primer to maintain reaction efficiency as the limiting primer concentration decreases mid-reaction. In this way, the linear phase of amplification can be extended, allowing more powerful quantification of the reaction progress than conventional real-time PCR methods. Therefore, the method can be used to detect SNPs with improved confidence.

In some embodiments, methods to detect SNPs are used in combination with molecular inversion probes (MIPs) as described in Hardenbol et al., Genome Res. 15(2) :269-275, 2005, Hardenbol, P. et al. Nature Biotechnology 21(6), 673-8, 2003; Faham M, et al. Hum Mol Genet. Aug 1; 10(16):1657-64, 2001; Maneesh Jain, Ph.D., et all. Genetic Engineering News V24: No. 18, 2004; and Fakhrai-Rad H, et al. Genome Res. Jul; 14(7):1404-12, 2004; and in U.S. Pat. No. 6,858,412. Universal tag arrays and reagent kits for performing such locus specific genotyping using panels of custom MIPs are available from Affymetrix. MIP technology involves the use enzymatic reactions that can score up to 10,000; 20,000, 50,000; 100,000; 200,000; 500,000; 1,000,000; 2,000,000 or 5,000,000 SNPs (target nucleic acids) in a single assay. The enzymatic reactions are insensitive to cross-reactivity among multiple probe molecules and there is no need for pre-amplification prior to hybridization of the probe with the genomic DNA. In any of the embodiments, the target nucleic acid(s) or SNPs are obtained from a single cell.

Thus, the present invention contemplate analyzing a sample using the MIP technology or oligonucleotide probes that are precircle probes i.e., probes that form a substantially complete circle when they hybridize to a SNP. The precircle probes comprise a first targeting domain that hybridizes upstream to a SNP position, a second targeting domain that hybridizes downstream of a SNP position, at least a first universal priming site, and a cleavage site. Once the probes are allowed to contact genomic DNA regions of interest (comprising SNPs to be interrogated) hybridization complex forms with a precircle probe and a gap at a SNP position region. Subsequently, ligase enzyme is used to “fill in” the gap or complete the circle. The enzymatic “gap fill” process occurs in an allele-specific manner The nucleotide added to the probe to fill the gap is complementary to the nucleotide base at the SNP position. Once the probe is circular, it may be separated from cross-reacted or unreacted probes by a simple exonuclease reaction. The circular probe is then cleaved at the cleavage site such that it becomes linear again. The cleavage site can be any site in the probe other than the SNP site. Linearization of the circular probe results in the placement of universal primer region at one end of the probe. The universal primer region can be coupled to a tag region. The tag can be detected using amplification techniques known in the art. The SNP analyzed can subsequently be detected by amplifying the cleaved (linearized) probe to detect the presence of the target sequence in said sample or the presence of the tag.

Another method contemplated by the present invention to detect SNPs involves the use of bead arrays as is commercially available by Illumina, Inc. and as described in U.S. Pat. Nos. 7,040,959; 7,035,740; 7033,754; 7,025,935, 6,998,274; 6, 942,968; 6,913,884; 6,890,764; 6,890,741; 6,858,394; 6,846,460; 6,812,005; 6,770,441; 6,663,832; 6,620,584; 6,544,732; 6,429,027; 6,396,995; 6,355,431 and US Publication Application Nos. 20060019258; 20050266432; 20050244870; 20050216207; 20050181394; 20050164246; 20040224353; 20040185482; 20030198573; 20030175773; 20030003490; 20020187515; and 20020177141; as well as Shen, R., et al. Mutation Research 573 70-82 (2005).

In some embodiments, SNP detection occurs by monitoring light scattering of nanoparticles, such as gold nanoparticles (Nanosphere, Inc.). These nanoparticles can be functionalized with oligonucleotide probes complementary to a given allele of a SNP. Single-base pair specificity for nucleic acid detection can be achieved due to assay reaction kinetics where gold nanoparticle probes, comprised of target-specific oligonucleotides, permit hybridization to target DNA over a very narrow temperature range. Hybridization causes a detectable alteration in light scattering by the nanoparticles, a parameter that can therefore be used to detect SNPs.

Those skilled in the art will know that a particular SNP, particularly when it occurs in a regulatory region of a gene such as a promoter, can be associated with altered expression of a gene. Altered expression of a gene can also result when the SNP is located in the coding region of a protein-encoding gene, for example where the SNP is associated with codons of varying usage and thus with tRNAs of differing abundance. Such altered expression can be determined by methods well known in the art, and can thereby be employed to detect such SNPs. Similarly, where a SNP occurs in the coding region of a gene and results in a non-synonymous amino acid substitution, such substitution can result in a change in the function of the gene product. Similarly, in cases where the gene product is RNA, such SNPs can result in a change of function in the RNA gene product. Any such change in function, for example as assessed in an activity or functionality assay, can be employed to detect such SNPs.

Genotype and/or SNP data can be stored in a data storage device. The data may be analyzed by a software comprising a computer-executable logic. These data can provided to uses at different locations such as by accessing a website, fax, email, mailed correspondence, automated telephone, telephone, video conference, or other methods for communication.

### Using SNPs to Predict Treatment Responsiveness

In some embodiments, the present disclosure provides for a method for determining responsiveness to an IL-6 inhibitor in a subject, comprising: (a) assaying SNP rs1800795 in a subject in need of a treatment of a disease with an IL-6 inhibitor; and (b) determining the responsiveness of said subject to said IL-6 inhibitor treatment, where the presence of G/C or G/G allele in said subject indicates low responsiveness. IL-6 inhibitors are detailed further herein.

SNP rs1800795 is located within the promoter region of IL-6. The C/C genotype for rs1800795 correlates with lower plasma IL-6 levels and platelet count compared to carriers of G/C or G/G alleles in healthy patients. Moreover, this polymorphism influences the amount of IL-6 production in response to inflammatory stimuli. These levels of IL-6 correspond in turn to the level of responsiveness to IL-6 inhibition and therefore analysis of SNP rs1800795 provides prediction of a response to treatment using one or more IL-6 inhibitors. In some embodiments, SNP rs1800795 is assayed to predict responders/non-responders of an IL-6 inhibitor-based treatment. SNP rs1800795 can also be used to examine the differential response of C/C versus G/C or G/G genotypes in determining or predicting dose response to an IL-6 inhibitor-based treatment.

In some embodiments, the present disclosure provides for a method for determining responsiveness to a JAK/STAT pathway inhibitor in a subject, comprising: (a) assaying SNP rs7574865 in a subject in need of a treatment of a disease with a JAK/STAT pathway inhibitor; and (b) determining the responsiveness of said subject to said JAK/STAT pathway inhibitor treatment, wherein the presence of G/T or T/T allele in said subject indicates low responsiveness. JAK/STAT pathway inhibitors are detailed further herein.

SNP rs7574865 is present within an intron of the gene encoding STAT4 and has a strong association with rheumatoid arthritis. SNP rs7574865 has been validated in populations of European, North American, and Asian descent with approximately 21.4-32.0% prevalence in these populations. The RA/SLE risk variant correlates with anti-citrullinate peptide antibodies (ACPA) presence in rheumatoid arthritis patients, and these antibodies are present in approximately 70% of rheumatoid arthritis patients. TNF-alpha activates the JAK/STAT pathway. STAT4 transmits signals for several gp130 classified cytokines (IL-6, IL-12, IL-23) and has many interactions with various other cytokines in the JAK/STAT pathway.

As a result of these interactions, assaying presence of G/T or T/T allele in SNP rs7574865 is useful in predicting responsiveness to therapeutics based on anti-TNF or anti-IL-6 treatments, as well as treatments targeting INF-alpha, JAK2, and JAK3.

### IL-6 Inhibitors

Interleukin-6 (IL-6) is a protein that is encoded by the IL-6 gene. IL-6 is an interleukin that acts as both a pro-inflammatory and anti-inflammatory cytokine. It is secreted by T cells and macrophages to stimulate immune response to trauma, especially burns or other tissue damage leading to inflammation. The pro-inflammatory activity of IL-6 makes it a useful target in treating inflammatory diseases and disorders.

In some embodiments, the present invention involves methods of treatment and determining responsiveness to treatment relating to IL-6 inhibitors.

IL-6 inhibitors may comprise any compound that inhibits or antagonizes the IL-6 pathway. Nucleic acids capable of functioning as pathway inhibitors are known in the art and include antisense nucleic acids, including DNA, RNA, or a nucleic acid analogues such as a peptide nucleic acid, locked nucleic acid, morpholino (phosphorodiamidate morpholino oligo), glycerol nucleic acid, or threose nucleic acid.

In addition, IL-6 inhibitors include peptides that block IL-6 signaling such as those described in any of U.S. Pat. Nos. 6,599,875; 6,172,042; 6,838,433; 6,841,533; and 5,210,075. Also, IL-6 inhibitors according to the invention may include p38 MAP kinase inhibitors such as those reported in US20070010529, given the role of p38 MAP kinase in production of cytokines such as IL-6. Further, IL-6 inhibitors according to the invention include the glycoalkaloid compounds reported in US20050090453 as well as other IL-6 antagonist compounds isolatable using the screening assays reported therein.

Other IL-6 inhibitors include antibodies, such as anti-IL-6 antibodies or antigen-binding fragments thereof, anti-IL-6 receptor alpha antibodies, anti-gp130 antibodies, and anti-p38 MAP kinase antibodies including (but not limited to) Actemra (Tocilizumab), Remicade, Zenapax, or any combination thereof. Other IL-6 inhibitors include portions or fragments of molecules involved in IL-6 signaling, such as IL-6, IL-6 receptor alpha, and gp130, which may be native, mutant, or variant sequence, and may optionally be coupled to other moieties (such as half-life-increasing moieties, e.g. an Fc domain). For example, an IL-6 inhibitor may be a soluble IL-6 receptor or fragment, a soluble IL-6 receptor:Fc fusion protein, a small molecule inhibitor of IL-6, an anti-IL-6 receptor antibody or antibody fragment or variant thereof, or antisense nucleic acid.

Other IL-6 inhibitors include avemirs, such as C326 (Silverman et al., Nat Biotechnol. 2005 December; 23(12):1556-61) and small molecules, such as synthetic retinoid AM80 (tamibarotene) (Takeda et al., Arterioscler Thromb Vasc Biol. 2006 May; 26(5): 1177-83).

Such IL-6 inhibitors may be administered by any means know in the art or described herein, including contacting a subject with nucleic acids which encode or cause to be expressed any of the foregoing polypeptides or antisense sequences.

### JAK/STAT Pathway Inhibitors

The JAK-STAT is a major signaling pathway for activating transcription in response to signals from components of the immune response, including interferon, interleukin, and growth factors. The JAK-STAT system consists of three main components: a receptor, JAK and STAT. JAK is short for Janus Kinase, and STAT is short for Signal Transducer and Activator of Transcription. Activation of the receptor activates the kinase function of JAK, which autophosphorylates itself The STAT protein then binds to the phosphorylated receptor. STAT is then phosphorylated and translocates into the cell nucleus, where it binds to DNA and promotes transcription of genes responsive to STAT. There are four JAK family members: Janus kinase 1 (JAK1), Janus kinase 2 (JAK2), Janus kinase 3 (JAK3), and Tyrosine kinase 2 (TYK2). JAK1 and JAK2 are involved in type II interferon (interferon-gamma) signaling, whereas JAK1 and TYK2 are involved in type I interferon signaling. JAK3 encodes Janus kinase 3, which is predominantly expressed in immune cells and transduces a signal in response to its activation via tyrosine phosphorylation by interleukin receptors.

As with IL-6, inhibition of the JAK-STAT pathway can be used to reduce an inflammation response and thereby treat an inflammatory disease or disorder.

A JAK/STAT pathway inhibitor may comprise an antibody against TNF-alpha, IL-6, INF-alpha, JAK2 or JAK3, or a fragment thereof.

Inhibition of TNF-alpha results in JAK-STAT inhibition, and has shown promise in treatment of inflammatory disorders. Known inhibitors of TNF-alpha include Cimzia CDP870 (certolizumab pegol), a pegylated Fab fragment of a humanized anti-TNF-alpha monoclonal antibody, and CNTO 148 (golimumab), a fully human IgGI-kappa anti-TNF-alpha monoclonal antibody.

Particularly preferred TNF-alpha inhibitors are biologic agents that have been approved by the FDA for use in humans in the treatment of rheumatoid arthritis, which agents include adalimumab (Humira), infliximab (Remicade) and etanercept (Enbrel). In other embodiments, the JAK/STAT pathway inhibitor is etanercept (described in WO 91/03553 and WO 09/406,476), infliximab (described in U.S. Pat. No. 5,656,272), CDP571 (a humanized monoclonal anti-TNF-alpha IgG4 antibody), CDP 870 (a humanized monoclonal anti-TNF-alpha antibody fragment), D2E7 (a human anti-TNF mAb), soluble TNF receptor Type I, or a pegylated soluble TNF receptor Type I (PEGs TNF-RI).

### Method of Treatment

In some embodiments, the present disclosure provides for a method for treating a subject, comprising:

(a) assaying SNP rs1800795 in a subject in need of a treatment of a disease with an IL-6 inhibitor; and
(b) administering to said subject a pharmaceutically effective amount of said IL-6 inhibitor, wherein the dosing regimen of said IL-6 inhibitor is based on the allele status of SNP rs1800795.

In some embodiments, the present disclosure provides for a method for treating a subject, comprising: (a) assaying SNP rs7574865 in a subject in need of a treatment of a disease with a JAK/STAT pathway inhibitor; and (b) administering to said subject a pharmaceutically effective amount of said JAK/STAT pathway inhibitor, wherein the dosing regimen of said JAK/STAT pathway inhibitor is based on the allele status of SNP rs7574865.

In some embodiments, dosing regimen is chosen based on results of SNP assay as described herein. SNPs can be used to predict drug efficacy and therefore, the presence of a given allele within a given SNP can inform a decision to administer a higher or lower dose of a treatment, accordingly. In some embodiments, the presence of G/C or G/G allele in SNP rs1800795 in a subject leads to prescription of a stronger dosing regimen for IL-6 inhibitor treatment. In some embodiments, the presence of G/T or T/T allele in SNP rs7574865 in a subject leads to prescription of a stronger dosing regimen for JAK/STAT pathway inhibitor treatment.

The dosing regimen can be selected or modified from one or more different locations such as through the use of a website, fax, email, mailed correspondence, automated telephone, telephone, video conference, or other methods for communication. A computer program comprising a computer-executable logic can be employed for transmission of data (e.g., regarding dosing regimen) over the internet.

Selection of the particular parameters of the treatment regimen can be based on known treatment parameters for a pathway inhibitor previously established in the art. For example, a non-limiting example of a treatment regimen for adalimumab (Humira) is 40 mg every other week by subcutaneous injection. A non-limiting example of a treatment regimen for etanercept (Enbrel) is 50 mg/week by subcutaneous injection. A non-limiting example of a treatment regimen for infliximab (Remicade) is 3 mg/kg by intravenous infusion at weeks 0, 2 and 6, then every 8 weeks. A treatment regimen can include administration of the pathway inhibitor alone or can include combination of the pathway inhibitor with other therapeutic agents, such as methotrexate (e.g., 10-20 mg/week) or prednisolone (e.g., 10 mg/week). Other suitable treatment regimens for the pathway inhibitors discussed herein will be readily apparent to the ordinarily skilled artisan based on prior studies of preferred administration parameters for the pathway inhibitor.

For the treatment of disease, the dosage and mode of administration can also be chosen according to known general criteria. The appropriate dosage will depend on the type of disease to be treated, as defined above, the severity and course of the disease, whether the treatment is administered for preventive or therapeutic purposes, previous therapy, the patient's clinical history and response to the treatment, and the discretion of the attending physician. The treatment is suitably administered to the patient at one time or over a series of treatments.

In some embodiments, IL-6 inhibitory antibody can be administered by intravenous infusion or by subcutaneous injections. Depending on the type and severity of the disease, about 1 μg/kg to about 50 mg/kg body weight (e.g. about 0.1-15 mg/kg/dose) of antibody can be an initial candidate dosage for administration to the patient, whether, for example, by one or more separate administrations, or by continuous infusion. A dosing regimen can comprise administering an initial loading dose of about 4 mg/kg, followed by a weekly maintenance dose of about 2 mg/kg of the anti-IL6 antibody. Another dosing regimen can comprise administering about 4 mg/kg, 5 mg/kg, 6 mg/kg, 7 mg/kg, or 8 mg/kg once every 4 weeks. Another dosing regimen can comprise administration of 80 mg, 160 mg, or 320 mg of anti-IL6 antibody twice over the course of 16 weeks. Other dosage regimens may be useful. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. The progress of this therapy can be readily monitored by conventional methods and assays and based on criteria known to the physician or other persons of skill in the art.

In some embodiments for treating cancer, the methods of treatment described herein may be combined with chemotherapy treatments known in the art. Sometimes, it may be beneficial to co-administer other anti-inflammatory treatments to the subject. A non-steroidal anti-inflammatory drug (NSAID) can be co-administered. Non-limiting examples of NSAIDs include acetylsalicylic acid (e.g., aspirin), ibuprofen (Motrin), naproxen (Naprosyn), indomethacin (Indocin), nabumetone (Relafen), tolmetin (Tolectin). An inhibitory composition may comprise acetaminophen (e.g., Tylenol), corticosteroids, or antimalarials (e.g., chloroquine, hydroxychloroquine), immunomodulating drug (e.g., azathioprine, cyclophosphamide, methotrexate, cyclosporine), an anti-B cell agent (e.g., anti-CD20 (e.g., rituximab), anti-CD22, Benlysta (BAFF/BLys), an anti-cytokine agent, anti-interleukin 10, anti-interleukin 6 receptor, anti-interferon alpha, anti-B-lymphocyte stimulator), an inhibitor of costimulation (e.g., anti-CD154, CTLA4-Ig (e.g., abatacept)), a modulator of B-cell anergy (e.g., LW 394 (e.g., abetimus)).

### Inflammatory Disorders

In some embodiments, the present disclosure is directed to methods of treatment or determining responsiveness to treatment for inflammatory diseases or disorders. In some embodiments, the subject is a human patient exhibiting symptoms of or diagnosed with an inflammatory disease or disorder. An inflammatory disease or disorder refers to immune and non-immune conditions associated with abnormal inflammation. Autoimmune disorders are inflammatory disorders and include, but are not limited to, Crohn's disease, ulcerative colitis, psoriasis, psoriatic arthritis, juvenile arthritis and ankylosing spondilitis, Other non-limiting examples of autoimmune disorders include autoimmune diabetes, multiple sclerosis, systemic lupus erythematosus (SLE), rheumatoid spondylitis, gouty arthritis, allergy, autoimmune uveitis, nephrotic syndrome, multisystem autoimmune diseases, autoimmune hearing loss, adult respiratory distress syndrome, shock lung, chronic pulmonary inflammatory disease, pulmonary sarcoidosis, pulmonary fibrosis, silicosis, idiopathic interstitial lung disease, chronic obstructive pulmonary disease, asthma, restenosis, spondyloarthropathies, Reiter's syndrome, autoimmune hepatitis, inflammatory skin disorders, vasculitis oflarge vessels, medium vessels or small vessels, endometriosis, prostatitis and Sjogren's syndrome.

Cancer is an example of an inflammatory disease. Many examples of cancers are known in the art. In some embodiments, the method is directed to the treatment of a cancer selected from Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia,Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, or any combination thereof.

Other non-immune diseases with etiological origins in inflammatory processes include atherosclerosis, and ischaemic heart disease.

In another embodiment, the disease is selected from general fatigue, exercise-induced fatigue, cancer-related fatigue, inflammatory disease-related fatigue, chronic fatigue syndrome, fibromyalgia, cancer-related cachexia, cardiac-related cachexia, respiratory-related cachexia, renal-related cachexia, age-related cachexia, rheumatoid arthritis, systemic lupus erythematosis (SLE), systemic juvenile idiopathic arthritis, psoriasis, psoriatic arthropathy, ankylosing spondylitis, inflammatory bowel disease (IBD), polymyalgia rheumatica, giant cell arteritis, autoimmune vasculitis, graft versus host disease (GVHD), Sjogren's syndrome, adult onset Still's disease, rheumatoid arthritis, systemic juvenile idiopathic arthritis, osteoarthritis, osteoporosis, Paget's disease of bone, osteoarthritis, multiple myeloma, Hodgkin's lymphoma, non-Hodgkin's lymphoma, prostate cancer, leukemia, renal cell cancer, multicentric Castleman's disease, ovarian cancer, drug resistance in cancer chemotherapy, cancer chemotherapy toxicity, ischemic heart disease, atherosclerosis, obesity, diabetes, asthma, multiple sclerosis, Alzheimer's disease, cerebrovascular disease, fever, acute phase response, allergies, anemia, anemia of inflammation (anemia of chronic disease), hypertension, depression, depression associated with a chronic illness, thrombosis, thrombocytosis, acute heart failure, metabolic syndrome, miscarriage, obesity, chronic prostatitis, glomerulonephritis, pelvic inflammatory disease, reperfusion injury, transplant rejection, graft versus host disease (GVHD), avian influenza, smallpox, pandemic influenza, adult respiratory distress syndrome (ARDS), severe acute respiratory syndrome (SARS), sepsis, and systemic inflammatory response syndrome (SIRS). In a preferred embodiment, the disease is selected from a cancer, inflammatory disorder, viral disorder, or autoimmune disorder. In a particularly preferred embodiment, the disease is arthritis, cachexia, and wasting syndrome.

Certain embodiments contemplate a human subject such as a patient that has been diagnosed as having or being at risk for developing or acquiring an inflammatory disorder. Certain other embodiments contemplate a non-human subject, for example a non-human primate such as a macaque, chimpanzee, gorilla, vervet, orangutan, baboon or other non-human primate, including such non-human subjects that can be known to the art as preclinical models, including preclinical models for inflammatory disorders. Certain other embodiments contemplate a non-human subject that is a mammal, for example, a mouse, rat, rabbit, pig, sheep, horse, bovine, goat, gerbil, hamster, guinea pig or other mammal. There are also contemplated other embodiments in which the subject or biological source can be a non-mammalian vertebrate, for example, another higher vertebrate, or an avian, amphibian or reptilian species, or another subject or biological source. In certain embodiments of the present invention, a transgenic animal is utilized. A transgenic animal is a non-human animal in which one or more of the cells of the animal includes a nucleic acid that is non-endogenous (i.e., heterologous) and is present as an extrachromosomal element in a portion of its cell or stably integrated into its germ line DNA (i.e., in the genomic sequence of most or all of its cells).

### Inhibitory Compositions

In some embodiments, the present disclosure involves methods of treatment using inhibitory compositions or methods of determining responsiveness to treatment using inhibitory compositions. An inhibitory composition may be any composition that inhibits a biological process, where non-limiting examples of biological processes include a protein function or signaling pathway. An inhibitory composition may therefore comprise a pathway inhibitor.

An inhibitory composition may comprise and antibody or fragment thereof. The term “antibody” as referred to herein includes whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, CH1, CH2, and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

The term “antigen-binding portion” of an antibody (or simply “antibody portion”), as used herein, refers to one or more fragments of an antibody that retain the ability to specifically bind to an antigen. It has been shown that the antigen binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, CL and CH1; (ii) a F(ab′)₂, fragment, a bivalent fragment comprising two Fab fragments linked by a disulfide bridge at the hinge region; (iii) a Fd fragment consisting of the V_{H} and CHI domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

An inhibitory composition may comprise a monoclonal antibody (mAb), a term that refers to a preparation of antibody molecules of single molecular composition. A monoclonal antibody composition displays a single binding specificity and affinity for a particular epitope. An inhibitory composition may comprise a chimeric antibody, which refers to an antibody in which the variable region sequences are derived from one species and the constant region sequences are derived from another species, such as an antibody in which the variable region sequences are derived from a mouse antibody and the constant region sequences are derived from a human antibody. Such chimeric antibodies can be prepared by standard recombinant technology well established in the art. For example, a nucleic acid encoding a V_{H} region from a mouse antibody can be operatively linked to a nucleic acid encoding the heavy chain constant regions from a human antibody and, likewise, a nucleic acid encoding a V_{L} region from a mouse antibody can be operatively linked to a nucleic acid encoding the light chain constant region from a human antibody.

An antibody may comprise a humanized antibody or humanized monoclonal antibody, wherein the humanized terminology refers to antibodies in which CDR sequences derived from the germline of a nonhuman mammalian species, such as a mouse, have been grafted onto human framework sequences. Additional framework region modifications may be made within the human framework sequences. Such humanized antibodies can be prepared by standard recombinant technology well established in the art. For example, nucleic acids encoding the CDRI, CD2 and CDR3 regions from a V_{H} region of a mouse antibody can be operatively linked to nucleic acids encoding the FRI, FR2, FR3 and FR4 regions of a human V_{H} region, and the entire “CDR-grafted” VH region can be operatively linked to nucleic acid encoding the heavy chain constant regions from a human antibody. Likewise, nucleic acids encoding the CDRI, CD2 and CDR3 regions from a V_{L} region of a mouse antibody can be operatively linked to nucleic acids encoding the FRI, FR2, FR3 and FR4 regions of a human V_{L} region, and the entire “CDR-grafted” region can be operatively linked to nucleic acid encoding the light chain constant region from a human antibody.

An antibody may comprise a human antibody, which refers to antibodies having variable regions in which both the framework and CDR regions are derived from human germline immunoglobulin sequences. Furthermore, if the antibody contains a constant region, the constant region also is derived from human germline immunoglobulin sequences. Human antibodies may include amino acid residues not encoded by human germline immunoglobulin sequences (e.g., mutations introduced by random or site-specific mutagenesis in vitro or by somatic mutation in vivo). A human monoclonal antibody may also be employed, referring to an antibody displaying a single binding specificity which has variable regions in which both the framework and CDR regions are derived from human germline immunoglobulin sequences. Human monoclonal antibodies can be produced by a hybridoma which includes a B cell obtained from a transgenic nonhuman animal, e.g., a transgenic mouse, having a genome comprising a human heavy chain trans gene and a light chain trans gene fused to an immortalized cell. The term “human monoclonal antibody”, as used herein, also includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom, (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. Such recombinant human antibodies, however, can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

An antibody may comprise a fusion protein or composite protein comprising a polypeptide of interest operatively linked to a constant region portion of immunoglobulin, typically the hinge, CH2 and CH3, domains of heavy chain constant region, more typically the human IgG1 hinge, CH2 and CH3 domains. The polypeptide of interest operatively linked to the Fc portion can be, for example, a full-length protein or only a portion of a full-length protein, such as one or more extracellular domains of a cell-surface protein.

An antibody may be modified. For example, pegylation of antibodies and antibody fragments of the invention may be carried out by any of the pegylation reactions known in the art, as described, for example, in the following references: Focus on Growth Factors 3:4-10 (1992); EP 0154316; and EP 0 401384 (each of which is incorporated by reference herein in its entirety). Preferably, the pegylation is carried out via an acylation reaction or an alkylation reaction with a reactive polyethylene glycol molecule (or an analogous reactive water-soluble polymer). A preferred water-soluble polymer for pegylation of the antibodies and antibody fragments of the invention is polyethylene glycol (PEG). As used herein, “polyethylene glycol” is meant to encompass any of the forms of PEG that have been used to derivatize other proteins, such as mono (CI-CIO) alkoxy- or aryloxy-polyethylene glycol.

Methods for preparing pegylated antibodies and antibody fragments of the invention will generally comprise the steps of (a) reacting the antibody or antibody fragment with polyethylene glycol, such as a reactive ester or aldehyde derivative of PEG, under conditions whereby the antibody or antibody fragment becomes attached to one or more PEG groups, and (b) obtaining the reaction products. It will be apparent to one of ordinary skill in the art to select the optimal reaction conditions or the acylation reactions based on known parameters and the desired result.

In some embodiments, antibodies or fragments thereof can be altered wherein the constant region of the antibody is modified to reduce at least one constant region-mediated biological effector function relative to an unmodified antibody. To modify an antibody of the invention such that it exhibits reduced binding to the Fc receptor, the immunoglobulin constant region segment of the antibody can be mutated at particular regions necessary for Fc receptor (FcR) interactions (see e.g., Canfield, S. M. and S. L. Morrison (1991) J. Exp. Med. 173:1483-1491; and Lund, J. et al. (1991) J. Immunol. 147:2657-2662). Reduction in FcR binding ability of the antibody may also reduce other effector functions which rely on FcR interactions, such as opsonization and phagocytosis and antigen-dependent cellular cytotoxicity.

An antibody or antibody portion of the invention can be derivatized or linked to another functional molecule (e.g., another peptide or protein). Accordingly, the antibodies and antibody portions of the invention are intended to include derivatized and otherwise modified forms of the antibodies described herein, including immunoadhesion molecules. For example, an antibody or antibody portion of the invention can be functionally linked (by chemical coupling, genetic fusion, noncovalent association or otherwise) to one or more other molecular entities, such as another antibody (e.g., a bispecific antibody), a detectable agent, a cytotoxic agent, a pharmaceutical agent, and/or a protein or peptide that can mediate association of the antibody or antibody portion with another molecule (such as a streptavidin core region or a polyhistidine tag).

One type of derivatized antibody is produced by crosslinking two or more antibodies (of the same type or of different types, e.g., to create bispecific antibodies). Suitable crosslinkers include those that are heterobifunctional, having two distinctly reactive groups separated by an appropriate spacer (e.g., m-maleimidobenzoyl-N-hydroxysuccinimide ester) or homobifunctional (e.g., disuccinimidyl suberate). Such linkers are available from Pierce Chemical Company, Rockford, Ill.

For administration to a subject, a pathway inhibitor typically is formulated into a pharmaceutical composition containing the TNF-alpha inhibitor and a pharmaceutically acceptable carrier. Therapeutic compositions typically must be sterile and stable under the conditions of manufacture and storage. Pharmaceutical compositions also can be administered in combination therapy, i.e., combined with other agents, such as other TNF-alpha inhibitors and/or other therapeutic agents, such as traditional therapeutic agents for the treatment of autoimmune disorders, such as rheumatoid arthritis (RA) or SLE.

The inhibitory composition may include one or more pharmaceutically acceptable carriers. As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound may be coated in a material to protect the compound from the action of acids and other natural conditions that may inactivate the compound.

The inhibitory composition may include one or more pharmaceutically acceptable salts. A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition also may include a pharmaceutically acceptable antioxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers that may be employed in the pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of microorganisms may be ensured both by sterilization procedures and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

An inhibitory composition of the present invention can be administered via one or more routes of administration using one or more of a variety of methods known in the art. As will be appreciated by the skilled artisan, the route and/or mode of administration will vary depending upon the desired results. A preferred route of administration, particularly for antibody agents, is by intravenous injection or infusion. Other preferred routes of administration include intramuscular, intradermal, intraperitoneal, subcutaneous, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intra sternal injection and infusion. Alternatively, a pathway inhibitor of the invention can be administered via a non-parenteral route, such as a topical, epidermal or mucosal route of administration, for example, intranasally, orally, vaginally, rectally, sublingually or topically.

### EXAMPLES

### Example 1

### Selection of Treatment Following Genotyping Using Molecular Inversion Probes for SNP analysis in Rheumatoid Arthritis Patients

Genotyping can be used to detect polymorphisms in SNP rs1800795 within the promoter region of IL-6. Genomic DNA can be obtained from a blood sample drawn from a patient diagnosed with Rheumatoid Arthritis. A Molecular Inversion Probe (MIP) with ends complementary to the sequences flanking SNP rs1800795 can then be added to the template genomic DNA, under conditions which permit hybridization of the MIPs to targets within the genomic DNA. Upon binding to the target, MIPs circularize, with a gap corresponding to the SNP. This gap can then be filled in according to the sequence using Klenow reaction, and ligated in a ligase reaction. Following an extension and ligation step, the mixture would be treated with exonuclease to remove all linear molecules and the tags of the surviving circular molecules would be amplified using PCR. The identity of a SNP within the genomic DNA is captured by the MIP probe, and may then be determined by quantitative PCR or sequencing methods.

Based on the results of the SNP analysis, a patient exhibiting the C/C genotype will not be administered tocilizumab therapy to inhibit IL-6, since the patient's disease is likely not to depend on the IL-6 pathway. In contrast, if a patient exhibits the G/G genotype, a higher dose of tocilizumab can be administered to inhibit a presumably higher level of IL-6 in the bloodstream.

### Example 2

### Selection of Anti-TNF Treatment Following SNP rs7574865 Analysis

A patient with physician diagnosed Rheumatoid Arthritis can be genotyped using analysis of SNP rs7574865 using the method of the invention. The patient may or may not be receiving anti-TNF therapy at the time of the SNP analysis. Genotyping may be performed with 10 ng DNA. Duplicate DNA samples may be genotyped as part of quality control assessments. Based on the genotype data, it can be determined if the patient has a G/T or T/T allele within SNP rs7574865. If the patient has the G/T or T/T allele within SNP rs7574865, anti-TNF treatment may not be recommended. Otherwise, the patient may be treated with anti-TNF therapeutic agents, such as etanercept, infliximab or adalimumab.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for determining responsiveness to an IL-6 inhibitor in a subject, comprising:
(a) assaying SNP rs1800795 for presence of G/C or G/G allele in a subject in need of a treatment of a disease with an IL-6 inhibitor; and
(b) determining the responsiveness of said subject to said IL-6 inhibitor treatment based on results from step (a).

2. A method for treating a subject, comprising:
(a) assaying SNP rs1800795 in a subject in need of a treatment of a disease with an IL-6 inhibitor; and
(b) administering a dosing regimen to said subject a pharmaceutically effective amount of said IL-6 inhibitor, based on the allele status of SNP rs180079.

3. The method of claim 1 or 2, wherein said disease is selected from the group consisting of:
rheumatoid arthritis, chronic juvenile arthritis, Crohn's disease, autoimmune diseases, diabetes and insulin sensitivity, neuroblastoma, systemic lupus erythematosus (lupus), and solid tumor cancer.

4. The method of any one of claims 1 to 3, wherein said IL-6 inhibitor comprises an antibody against IL-6 or a fragment thereof.

5. The method of claim 4, wherein said antibody is tocilizumab.

6. A method for determining responsiveness to a JAK/STAT pathway inhibitor in a subject, comprising:
(a) assaying SNP rs7574865 for presence of G/T or T/T allele in a subject in need of a treatment of a JAK/STAT pathway inhibitor; and
(b) determining responsiveness for treatment based on results from step (a).

7. A method for treating a subject, comprising:
(a) assaying SNP rs7574865for allele status in a subject in need of a treatment of a disease with a JAK/STAT pathway inhibitor; and
(b) administering to said subject a pharmaceutically effective amount of said JAK/STAT pathway inhibitor in a dose dependent on results from step (a).

8. The method of claim 6 or 7, wherein said disease is selected from the group consisting of:
rheumatoid arthritis, chronic juvenile arthritis, Crohn's disease, systemic lupus erythematosus (SLE), Sjorgen's syndrome, and associated inflammatory disease.

9. The method of any one of claims 6 to 8, wherein said JAK/STAT pathway inhibitor comprises an antibody against TNF, IL-6, INF-alpha, JAK2 or JAK3, or a fragment thereof.

10. The method of claim 9, wherein said antibody is Humira or Remicade.
